(19) United States
(12) Patent Application Publication
(10) Pub. No.: US 2001/0031940 A1
(43) Pub. Date: Oct. 18, 2001

(54) Device for administering implants

(76) Inventor: Hans-Joachim Loos, Ginsheim-Gustavsburg 1 (DE)

Correspondence Address: Finnegan, Henderson, Farabow,, Garrett & Dunner, L.L.P., 1300 I Street, N.W., Washington, DC, 20005-3315, US

(21) Appl. No.: 09/814,121

(22) Filed: Mar. 22, 2001

(30) Foreign Application Priority Data
Mar. 23, 2000 (DE) 10014518.3-44

(51) Int. Cl. A61F 13/20; A61M 31/00

(52) U.S. Cl. 604/15

(57) ABSTRACT

The present invention is related to a device for administering implants. The device is a syringe-like device having a plunger, an injection cannula, and an active substance container therebetween. The active substance container includes two retaining elements for preventing inadvertent dispensing of an implant. The retaining elements are flexible, and may be O-rings.

### BACKGROUND OF THE INVENTION

1. Field of the Invention

The present invention relates to a syringe type device for administering implants to a patient.

2. Description of the Prior Art

Syringe type devices for administering implants are shown, for example, in DE 38 02 158 A1 and DE 43 20 754 A1. However, these conventional devices have several drawbacks. Some devices, such as that disclosed in DE 38 02 158 A1, do not have any structure to retain the implants within the device prior to implantation. This results in implants inadvertently dropping out of the device prior to implantation. Often, such implants can no longer be used because they are no longer sterile, resulting in increased costs to the hospital and patient. Additionally, new implants must be loaded into the administering device, requiring extra time and thus reducing efficiency of the operation. Other devices, such as those shown in DE 43 20 754 A1, include complex mechanical structures for holding the implants in place prior to administering them. Due to the complexity of these structures, the device sometimes jams, making delivery of the implant unreliable. The structures are also somewhat rigid and may cause damage to the implant as it passes through the structure, for example, by deforming or abrading the implant.

### SUMMARY OF THE INVENTION

Accordingly, the present invention is directed to a method and device for administering an implant which provides advantages and obviates a number of problems in earlier devices for administering implants.

To achieve the object and in accordance with the purpose of the invention, as embodied and broadly described herein, the invention comprises a device for administering an implant, including a plunger, an injection cannula, and an active substance container, the active substance container being located between the plunger and the injection cannula, the container including at least one flexible retaining element for preventing inadvertent dispensing of an implant.

To achieve another object in the accordance with the purpose of the invention, as embodied and broadly described herein, the invention comprises a device for administering an implant including a plunger, an injection cannula, and an active substrate container, the active substrate container being located between the plunger and the injection cannula, the container including two retaining elements, one of said retaining elements being positioned on each end of said container.

It is to be understood that both the foregoing general description and the following detailed description are exemplary and explanatory only and are not restrictive of the invention as claimed.

The accompanying drawings, which are incorporated in and constitute a part of this specification, illustrate one embodiment of the invention and together with the description, serve to explain the principles of the invention.

### BRIEF DESCRIPTION OF THE DRAWINGS

In the drawings,

FIG. 1 is a cross-sectional side view of an embodiment of the device for administering implants according to the invention; and

FIG. 2 is an exploded cross-sectional side view of the device of FIG. 1.

### DESCRIPTION OF THE PREFERRED EMBODIMENT

Reference will now be made in detail to a present embodiment of the invention, an example of which is illustrated in the accompanying drawings. Wherever possible, the same reference numbers will be used throughout the drawings to refer to the same or like parts.

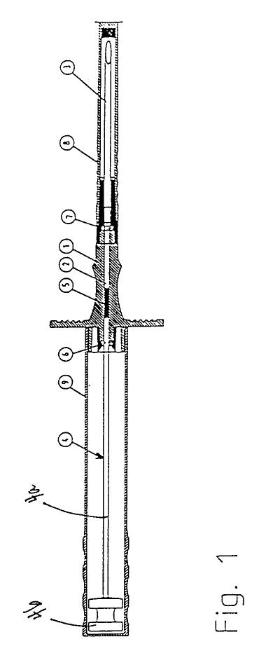

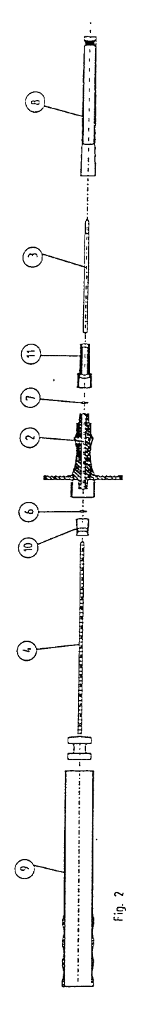

According to the present invention, and as embodied in FIGS. 1 and 2, the device for administering implants includes an active substance container, an injection cannula, and a plunger.

As shown in FIG. 1, an active substance container 1 is provided to hold the implants prior to implantation. Active substance container 1 is positioned between a plunger 4 and an injection cannula 3. Container 1 includes at least a portion of a plunger channel 2 which passes through active substance container 1 and is in communication with an injection cannula 3.

Plunger channel 2 passes through active substance container 1 and through 1 injection cannula 3. Plunger channel 2 is configured to receive a plunger 4. Plunger 4 has a body portion 4a for moving through the plunger channel 2 and pushing the implant 5 ahead of it through the plunger channel 2. Plunger 4 also includes a head portion 4b configured to allow a user to depress the plunger 4 to cause the plunger body portion 4a to move through the plunger channel 2. A portion of plunger channel 2 passes through the active substance container 1 and is sized to hold at least one implant 5, and preferably is sized to hold more than one implant 5. In one embodiment, implant 5 is rod-shaped, but implants of other shapes, such as spherical or oval, may also be used.

Active substance container 1 includes a holding structure to ensure that implant 5, when contained within active substance container 1 cannot inadvertently fall out of the container 1. As drawn, the holding structure includes first and second retaining elements 6, 7. As shown in FIG. 2, a first retaining element 6 is preferably placed at an upper or first end of the container 1, located between plunger 4 and container 1. Retaining element 6 is preferably fixed to the container by an adapter cap 10. A second retaining element 7 is placed at an opposite end of container 1, between the container 1 and injection cannula 3. In this embodiment, retaining elements 6, 7 are O-rings, each O-ring having an internal diameter smaller in size than a diameter of the plunger channel 2. O-rings 6, 7 are placed to be co-axial with plunger channel 2, such that a portion of each O-ring protrudes into plunger channel 2, above and below the active substance container 1, respectively. By protruding into plunger channel 2, retaining elements 6, 7 form an obstacle that the implant must pass to exit the active substance container 1.

In addition, although O-rings are one embodiment of the retaining element, it is possible to use other structures. O-rings, preferably made of a flexible material such as a silicone elastomer or a synthetic rubber, were chosen due to the fact that they are easily deformable. Thus, when pressure is applied to the O-ring, as in the case where the plunger is pushing an implant 5 through plunger channel 2 past retaining element 7 into injection cannula 3, the O-ring will tend to give or deform and allow the implant to pass. A more rigid structure might abrade or damage the implant instead of allowing it easy passage. Thus, any structure used as a retaining element must be flexible and/or easily deformable so as to yield to the implant when under pressure, such as when the plunger 4 is moving the implant 5 through the plunger channel 2. Other examples of such suitable materials/retaining elements include rings or “bumps” made of a gel or viscous material which is easily deformable. Other suitable materials include silicone caotchouc, chlorbutyl caotchouc, brombutyl caotchouc, and LD (low density)polyethylene. Silicone caotchouc is a preferred material.

The device may also include protective caps for covering the injection cannula and the plunger. As shown in FIGS. 1 and 2, plunger 4 may be covered with a plunger cap 9 and injection cannula 3 may be covered by a cannula protective cap 8. Such caps are desirable as they help to maintain the sterility of the device for administering implants.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A device for administering implants, comprising:
a plunger;
an injection cannula; and
an active substance container, the active substance container being located between the plunger and the injection cannula, the container including two flexible retaining elements for preventing inadvertent dispensing of an implant.

2. The device of claim 1, wherein a plunger channel passes through the active substance container into the injection cannula.

3. The device of claim 2, wherein the retaining elements partially extend into the plunger channel.

4. The device of claim 3, wherein one of the flexible retaining elements is positioned adjacent one end of the container, between the container and the injection cannula, and the other retaining element is positioned adjacent the other end of the container.

5. The device of claim 4, wherein the retaining elements are O-rings.

6. The device of claim 4, wherein the retaining elements are made from one of a silicone elastomer and a synthetic rubber.

7. The device of claim 1, wherein one of the retaining elements is positioned between the active substance container and the injection cannula.

8. The device of claim 1, wherein the retaining elements are O-rings.

9. The device of claim 1 , wherein the retaining elements are made from one of a silicone elastomer and a synthetic rubber.

10. A device for administering implants, comprising:
a plunger;
an injection cannula; and
an active substance container, the active substance container being located between the plunger and the injection cannula, the container including two retaining elements, one of said retaining elements being positioned on each end of said container.

11. The device of claim 10, wherein the retaining elements are flexible.

12. The device of claim 10, wherein a plunger channel passes through said container into said injection cannula, and wherein said retaining elements partially extend into said channel.

13. The device of claim 10, wherein the retaining elements are O-rings.

14. The device of claim 10, wherein the retaining elements are made from on e of a silicone elastomer and a synthetic rubber.
